(19) United States
(12) Patent Application Publication
(10) Pub. No.: US 2024/0428414 A1
(43) Pub. Date: Dec. 26, 2024

(54) METHOD AND SYSTEM FOR AUTOMATED ECHOCARDIOGRAPHIC DETECTION

(71) Applicants: Northwestern University, Evanston, IL (US); The Board of Trustees of the Univ. of Illinois, Urbana, IL (US)

(72) Inventors: Vinesh Appadurai, Chicago, IL (US); Akhil Narang, Highland Park, IL (US); Sanjiv J. Shah, Chicago, IL (US); Brody Slostad, Chicago, IL (US); James David Thomas, Chicago, IL (US); Mayank Kansal, Chicago, IL (US)

(21) Appl. No.: 18/750,047

(22) Filed: Jun. 21, 2024

Related U.S. Application Data
(60) Provisional application No. 63/522,204, filed on Jun. 21, 2023.

(51) Int. Cl. G06T 7/00; A61B 8/08; G06V 10/764; G06V 20/50

(52) U.S. Cl. CPC G06T 7/0014; A61B 8/0883; G06V 10/764; G06V 20/50; G06T 2207/30048; G06V 2201/031

(57) ABSTRACT

A system to distinguish subtypes of cardiac amyloidosis includes a memory configured to store an echocardiographic image of at least a portion of a heart. The system also includes a processor operatively coupled to the memory. The processor is configured to analyze the echocardiographic image to identify a region of interest of the heart. The processor also determines a septal reflectivity ratio of the region of interest. The processor also determines, based at least in part on the septal reflectivity ratio, a subtype of cardiac amyloidosis present in the region of interest.

### CROSS-REFERENCE TO RELATED APPLICATION

The present application claims the priority benefit of U.S. Provisional Patent App. No. 63/522,204 filed on Jun. 21, 2023, the entire disclosure of which is incorporated by reference herein.

### BACKGROUND

Cardiac amyloidosis refers to a heart condition in which abnormal proteins become deposited in the heart tissue. These abnormal protein deposits make it difficult for the heart to function properly and can result in serious complications or death without treatment. One subtype of cardiac amyloidosis is referred to as transthyretin amyloidosis, which is a disorder in which clumps of irregular proteins are produced by the liver and travel to the heart where they then accumulate. Another subtype of cardiac amyloidosis is referred to as light chain (or primary) amyloidosis. Light chain amyloidosis is a protein misfolding and metabolism disorder in which insoluble fibrils are deposited in various tissues of the heart. While both transthyretin amyloidosis and light chain amyloidosis are heart conditions resulting from abnormal protein buildups, the treatment strategies for these two conditions differs significantly.

### SUMMARY

An illustrative system to distinguish subtypes of cardiac amyloidosis includes a memory configured to store an echocardiographic image of at least a portion of a heart. The system also includes a processor operatively coupled to the memory. The processor is configured to analyze the echocardiographic image to identify a region of interest of the heart. The processor also determines a septal reflectivity ratio of the region of interest. The processor also determines, based at least in part on the septal reflectivity ratio, a subtype of cardiac amyloidosis present in the region of interest.

In an illustrative embodiment, the echocardiographic image comprises a view of a left ventricle in the parasternal long axis. In another embodiment, the view of the echocardiographic image is captured at end diastole. In another embodiment, the region of interest is bounded by an anterior septal myocardial wall of the heart and a posterior lateral myocardial wall of the heart. In another illustrative embodiment, the region of interest includes fibrils containing microcalcification clusters.

In another embodiment, the processor is further configured to determine a luminance of the region of interest. The processor is configured to determine a first pixel intensity of an anterior septal myocardial wall of the heart and a second pixel intensity of a posterior lateral myocardial wall of the heart based at least in part on the luminance. The processor divides the first pixel intensity by the second pixel intensity to determine the septal reflectivity ratio. The processor also compares the septal reflectivity ratio to a threshold value to determine the subtype of cardiac amyloidosis. In one embodiment, the threshold value is 1.23. In an illustrative embodiment, the processor distinguishes between transthyretin cardiac amyloidosis and light chain cardiac amyloidosis based on the comparison to the threshold value.

An illustrative method to distinguish subtypes of cardiac amyloidosis includes receiving, at a memory of a computing device, an echocardiographic image of at least a portion of a heart. The method also includes analyzing, by a processor of the computing device, the echocardiographic image to identify a region of interest of the heart. Eth method also includes determining, by the processor, a septal reflectivity ratio of the region of interest. The method further includes determining, by the processor and based at least in part on the septal reflectivity ratio, a subtype of cardiac amyloidosis present in the region of interest.

In an illustrative embodiment, the method includes capturing the echocardiographic image such that the echocardiographic image includes a view of a left ventricle in the parasternal long axis, where the echocardiographic image is captured at end diastole. In another embodiment, determining the region of interest includes identifying a region of the heart that is bounded by an anterior septal myocardial wall of the heart and a posterior lateral myocardial wall of the heart. The region of interest includes fibrils containing microcalcification clusters.

The method can also include determining, by the processor, a first pixel intensity of pixels corresponding to an anterior septal myocardial wall of the heart and a second pixel intensity of pixels corresponding to a posterior lateral myocardial wall of the heart. The method also includes dividing, by the processor, the first pixel intensity by the second pixel intensity to determine the septal reflectivity ratio. The method further includes comparing, by the processor, the septal reflectivity ratio to a threshold value to determine the subtype of cardiac amyloidosis. In an illustrative embodiment, the method includes distinguishing, by the processor, between transthyretin cardiac amyloidosis and light chain cardiac amyloidosis based on the comparison to the threshold value.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1 depicts determination of the septal reflectivity ratio based on image analysis in accordance with an illustrative embodiment.

FIG. 2A depicts measuring septal reflectivity ratio with examples of optimal (left) and suboptimal (right) parasternal long axis images in accordance with an illustrative embodiment.

FIG. 2B depicts measuring the septal reflectivity ratio with appropriately selected regions of interest in an optimal image in accordance with an illustrative embodiment.

FIG. 3 is a table that depicts the clinical, demographic and laboratory characteristics of the study population in accordance with an illustrative embodiment.

FIG. 4 is a table that depicts relevant echocardiography parameter findings across the 4 cohorts in accordance with an illustrative embodiment.

FIG. 5A is a chart that depicts the mean septal reflectivity ratio for the 4 cohorts in the study in accordance with an illustrative embodiment.

FIG. 5B is a chart that depicts the mean increased wall thickness (IWT) for the 4 cohorts in the study in accordance with an illustrative embodiment.

FIG. 5C is a chart that depicts the mean global longitudinal strain (GLS) for the 4 cohorts in the study in accordance with an illustrative embodiment.

FIG. 5D is a chart that depicts the mean mass to strain ratio (MSR) for the 4 cohorts in the study in accordance with an illustrative embodiment.

FIG. 5E is a chart that depicts the mean relative apical sparing ratio (RASR) for the 4 cohorts in the study in accordance with an illustrative embodiment.

FIG. 5F is a chart that depicts the mean septal apical to base ratio (SAB) for the 4 cohorts in the study in accordance with an illustrative embodiment.

FIG. 6 is a table that depicts receiver operating characteristic curve analysis for SRR versus strain-based parameters differentiating ATTR-CA from AL-CA in accordance with an illustrative embodiment.

FIG. 7 is a graph that depicts ROC curves for differentiating ATTR-CA from all other cohorts and that compares SRR against other important strain-based parameters in accordance with an illustrative embodiment.

FIG. 8 is a table that depicts receiver operating characteristic curve analysis for SRR versus strain-based parameters differentiating ATTR-CA from other cohorts in accordance with an illustrative embodiment.

FIG. 9 is a graph that depicts ROC curves for differentiating ATTR-CA from AL-CA by comparing SRR against other important strain-based parameters in accordance with an illustrative embodiment.

FIG. 10 is a block diagram of components included in an echocardiographic detection system for distinguishing subtypes of cardiac amyloidosis in accordance with an illustrative embodiment.

### DETAILED DESCRIPTION

Cardiac amyloidosis (CA) is an infiltrative cardiomyopathy occurring secondary to the deposition of misfolded proteins in the myocardial extracellular space. Cardiac amyloidosis carries significant morbidity and mortality, and delineating CA subtypes is integral to ensuring appropriate disease modifying therapies are initiated as early as possible. Although there are around 30 different types of amyloid protein that can deposit in myocardium, CA can broadly be divided into two subtypes. Specifically, the deposition of proliferative immunoglobulin monoclonal proteins resulting in light chain amyloidosis (AL-CA) and the deposition of misfolded thyroxine transporter protein resulting in transthyretin-related cardiac amyloidosis (ATTR-CA). These two subtypes of CA have significantly different outcome trajectories and are by far the most common forms of cardiac amyloidosis. As a result, imaging modalities that can differentiate myocardial alterations between CA subtypes are important for accurate diagnosis and management.

Echocardiography is the first-line imaging modality utilized to investigate the presence of CA due to its accessibility, safety profile, and wealth of hemodynamic and structural information. Post-processing speckle-tracking echocardiography (STE) can reveal reductions in basal longitudinal contraction secondary to amyloid infiltration and demonstrate an apical-sparing strain pattern suggestive of CA. However, this pattern lacks specificity for CA sub-types and therefore requires down-stream confirmatory testing. Furthermore, subsequent non-invasive testing such as cardiac magnetic resonance imaging (MRI) or nuclear scans utilizing either diphosphono-1,2-propanodicarboxylic acid or pyrophosphate (PYP) along with serum and urine immunofixation are needed to confirm CA and define whether ATTR-CA or AL-CA is present due to the aforementioned non-specific nature of STE strain plots. For that reason, developing an echocardiographic, tissue-specific tool to determine whether ATTR amyloidosis may be present over AL amyloidosis would provide significant value to the current CA diagnostic landscape.

In comparison to AL-CA, ATTR-CA is unique in that calcium-binding tracers used in nuclear scans can detect the presence of transthyretin deposition in the myocardium. Furthermore, autopsy analysis of whole hearts with cardiac amyloidosis have confirmed the presence of microcalcification clusters. These clusters are significantly increased in the ATTR-CA affected hearts when compared to the AL-CA affected hearts, with microcalcification predilection to the left ventricle, but in particular the interventricular septum. Moreover, multi-modality imaging has supported the progression of ATTR deposition from a base to apical gradient within the left ventricle. Based on these characteristics, the inventors sought to develop a novel application in CA patient echocardiograms via relative pixel intensity quantification. The aim was to develop a technique to differentiate ATTR-CA from AL-CA in patients presenting with proven CA and known phenocopies of CA.

As part of developing the differentiation technique, the inventors conducted a retrospective review of patients referred to a medical facility for evaluation of suspected ATTR-CA and AL-CA based on echocardiographic and clinical features. Ground truth of ATTR-CA was confirmed with either endomyocardial biopsy or positive 99m technetium pyrophosphatc (99mTc-PYP), in addition to positive genetic testing for ATTR-CA. Bone marrow biopsy and endomyocardial biopsy were utilized to confirm a diagnosis of AL-CA.

Established echocardiographic phenocopies of CA, such as hypertrophic cardiomyopathy (HCM) and advanced chronic kidney disease (CKD) patients were included as comparator groups. They were identified utilizing an institutional echocardiographic database over a 12-month period using search terms “hypertrophic cardiomyopathy” and “chronic kidney disease” or “dialysis”. Furthermore, the diagnosis of hypertrophic cardiomyopathy was defined as left ventricular hypertrophy (LVH) ≥15 millimeters (mm) anywhere in the left ventricle (LV) wall in the absence of any other identifiable cause such as hypertension or valve disease, and was confirmed by a cardiologist in the heart failure with preserved ejection fraction clinic. Similarly, the diagnosis of CKD for the purposes of this study was any patient with stage 4 or greater CKD (GFR<30 mL/min/1.73 m²) or on chronic dialysis with a confirmed diagnosis by a nephrologist.

As part of the analysis, echocardiography studies were performed by trained sonographers using commercially available ultrasound platforms (i.e. Philips, GE, and Siemens). A standardized imaging protocol based on the American Society of Echocardiography (ASE) guidelines was utilized. This included ejection fraction (EF) using the Simpson's biplane method from the apical two- and four-chamber windows, left atrial volume index using a biplane area-length formula, end-diastolic interventricular septal (IVS), posterior wall (PW) thickness, and LV internal dimension in diastole. LV mass index was calculated via the Devereaux method. Relative wall thickness (RWT) was calculated as (IVS+PWT)/LV diastolic diameter. Diastolic parameters, including peak early (E) and late (A) diastolic mitral inflow velocity and its ratio (E/A), deceleration time (DT), and average of the medial and lateral mitral annular diastolic velocities (e′), were also measured according to ASE guidelines.

Longitudinal strain measurements were performed offline using fully automated software (TomTec TTA Version 2.41) across three standard apical views and are presented as whole percentages (not negative values). Using the data, a ‘bull's-eye’ plot demonstrating segmental strain values was generated. Echocardiographic images were considered unacceptable if two or more segments did not track adequately after two manual adjustments of the endocardial borders. Strain values from all segments were averaged to obtain a global longitudinal strain value. The apex-to-base gradient in regional longitudinal strain was calculated using absolute strain values as well as relative apical strain. Echocardiographic components including relative wall thickness (RWT), tricuspid annular plane systolic excursion (TAPSE), E-wave/e′-wave (E/e′), global longitudinal strain (GLS), and septal systolic apical-to-base ratio (SAB) were recorded to calculate CA-based scores such as the increased wall thickness (IWT) score. Similarly, the LV mass to strain ratio (MSR), LVEF to strain ratio, and relative longitudinal apical sparing ratio (RASR) were evaluated.

The study focused on relative pixel intensity quantification. Patients with at least 75% of both their anterior septal and posterior lateral walls visible on parasternal long axis images with adequate endocardial border definition and uniform gain settings at all image depths were included in the study. These characteristics allow for identification and distinguishing of the walls from blood pool and adjacent structures. Patients with rib shadowing or variable gain settings that limited visualization of the anterior or posterior walls were excluded.

The analyzed images included a still frame of the LV in the parasternal long axis at end diastole. Pixel intensity was then measured. Pixel intensity refers to the brightness or luminance of the light emitted by a digital display, corresponding to the anterior septal and posterior lateral myocardial walls at end diastole in the parasternal long-axis view. This was performed using the free, publicly available pixel intensity quantification software, ImageJ (developed by the National Institutes of Health). In alternative embodiments, a different type of pixel intensity quantification software may be used. The mean pixel intensity values ranged from 0 to 255, where 0 represents a black pixel and 255 represents a white pixel. Manual annotation was used to segment the entire anterior septal wall and posterior lateral wall that were visible in the parasternal long-axis view. No significant differences in gain related to depth and time-gain compensation were noted within each image. Pixel densities of the left ventricular blood pool immediately adjacent to both walls and in the right ventricular blood pool were recorded and compared and showed no significant differences in gain within each image at the depth of the anterior septal and posterior lateral walls.

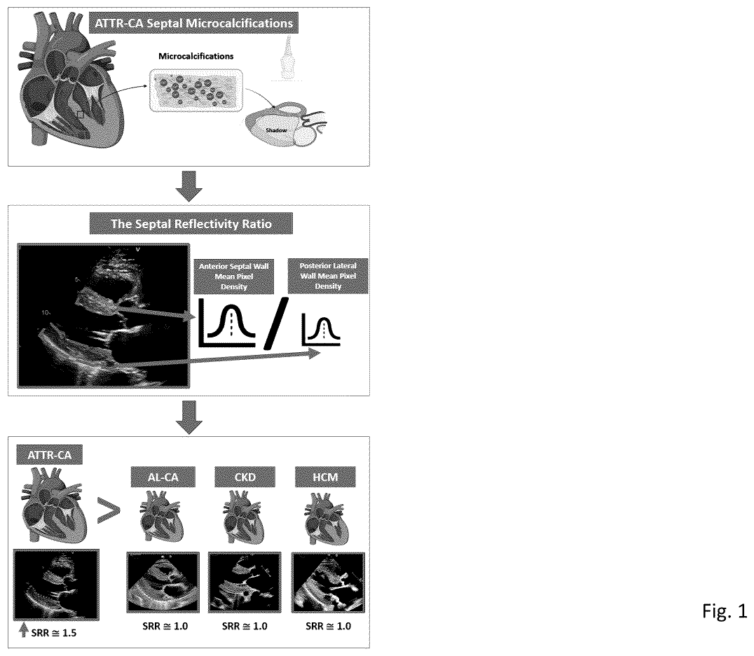

The anterior septal wall pixel intensity divided by the posterior lateral wall pixel intensity resulted in a value that is referred to as the septal reflectivity ratio (SRR). FIG. 1 depicts determination of the septal reflectivity ratio based on image analysis in accordance with an illustrative embodiment. FIG. 1 uses the following acronyms: ATTR-CA=transthyretin-related cardiac amyloidosis; AL-CA=light chain cardiac amyloidosis; CKD=chronic kidney disease; and HCM=hypertrophic cardiomyopathy. The septal reflectivity ratio takes advantage of the of preferential deposition of ATTR-CA fibrils containing microcalcification clusters in the ventricular septum, as shown in the upper panel of FIG. 1. These microcalcification clusters cause acoustic shadowing of the posterior lateral wall (upper panel of FIG. 1). The septal reflectivity ratio is obtained by measuring the mean pixel intensity of the visible anterior septal wall and dividing this by the mean pixel intensity of the visible posterior lateral wall in end-diastole in the parasternal long axis still frame (middle panel of FIG. 1). End diastole, as used herein, refers to a state at which the heart has performed diastolic filling and is ready for contraction. End diastole can also refer to an amount (e.g., volume) of blood in one or both of the heart ventricles immediately prior to contraction of the heart. The mean septal reflectivity ratio in patients with ATTR-CA (≅1.5) was significantly higher than patients with AL-CA, CKD and HCM (all≅1.0; lower panel of FIG. 1).

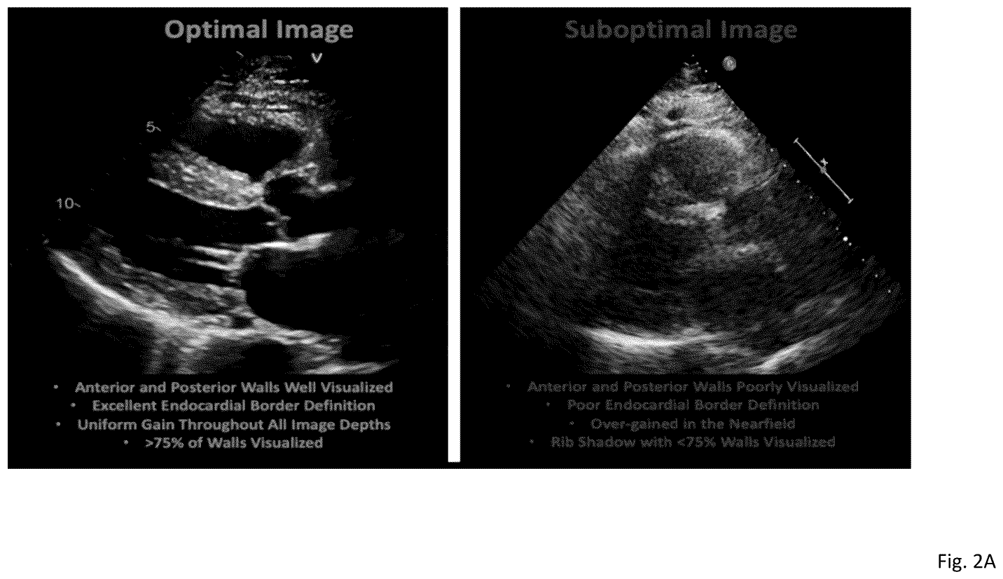

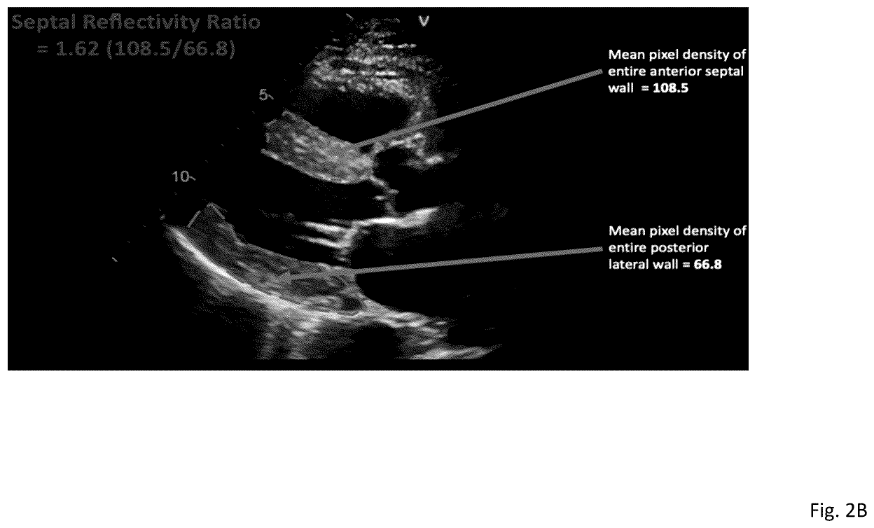

FIG. 2A depicts measuring septal reflectivity ratio with examples of optimal (left) and suboptimal (right) parasternal long axis images in accordance with an illustrative embodiment. FIG. 2B depicts measuring the septal reflectivity ratio with appropriately selected regions of interest in an optimal image in accordance with an illustrative embodiment. It is noted that two trained echocardiographers, who were blinded to the clinical data, independently analyzed and re-measured the SRR for 30 randomly assigned patients to assess for intra- and inter-class reliability.

A statistical analysis was also conducted as part of the study. Demographic, laboratory, and imaging data were collected and analyzed with data presented as mean and standard deviation (SD) or as percentages. Statistical analysis was performed using IBM SPSS (version 29, Armonk, New York). For comparisons between study subgroups, differences in categorical variables were analyzed using the χ2 test or when appropriate, the Fisher exact test, while differences in continuous variables were analyzed using a 1-way ANOVA with post hoc Bonferroni correction. Area under the receiver operating characteristic (ROC) curve was used to determine the accuracy of septal-reflectivity ratio for identifying ATTR amyloidosis. The Delong test and Youden's index were used to compare significance between ROC curves and determine a suitable diagnostic cut-off respectively. Expert reader analysis was compared using Cohen's Kappa statistic. In addition, intraclass correlation was used to evaluate reproducibility of septal-reflectivity ratio measurements.

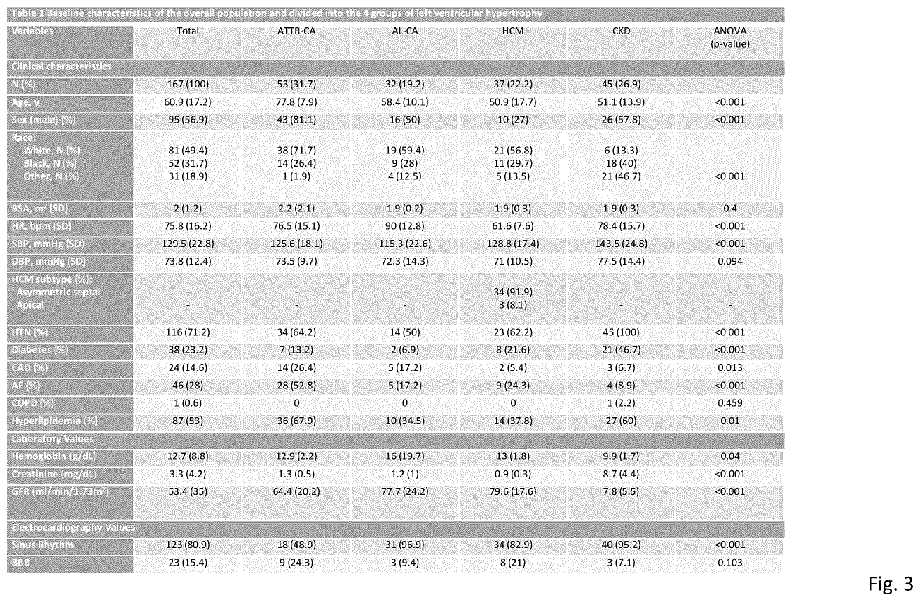

FIG. 3 is a table that depicts the clinical, demographic and laboratory characteristics of the study population in accordance with an illustrative embodiment. There were a total of 205 patients identified, of which 38 patients did not have appropriate imaging for performing the SRR measurements. As a result, 167 patients remained in the final analysis cohort. Of these, 53 patients (31.7%) had transthyretin-related cardiac amyloidosis (ATTR-CA), 32 patients (19.2%) had light-chain related cardiac amyloidosis (AL-CA), 37 patients (22.2%) had hypertrophic cardiomyopathy (HCM), and 45 patients (26.9%) had advanced CKD. The mean age of the total population was 60.9 (+/−17.2) years. The ATTR-CA patients were significantly older (77.8+/−7.9 years) compared to the other 3 groups. Of the total population, 56.9% were male and this varied across the subgroups with 81.1% of patients within the ATTR-CA cohort being male to only 27% within the HCM cohort. There were significantly more hypertensives within the advanced CKD cohort and significantly more patients had some form of atrial fibrillation (AF) within the ATTR-CA cohort compared to other groups. Sinus rhythm was predominately observed within the advanced CKD cohort compared to only 48.9% within the ATTR-CA cohort.

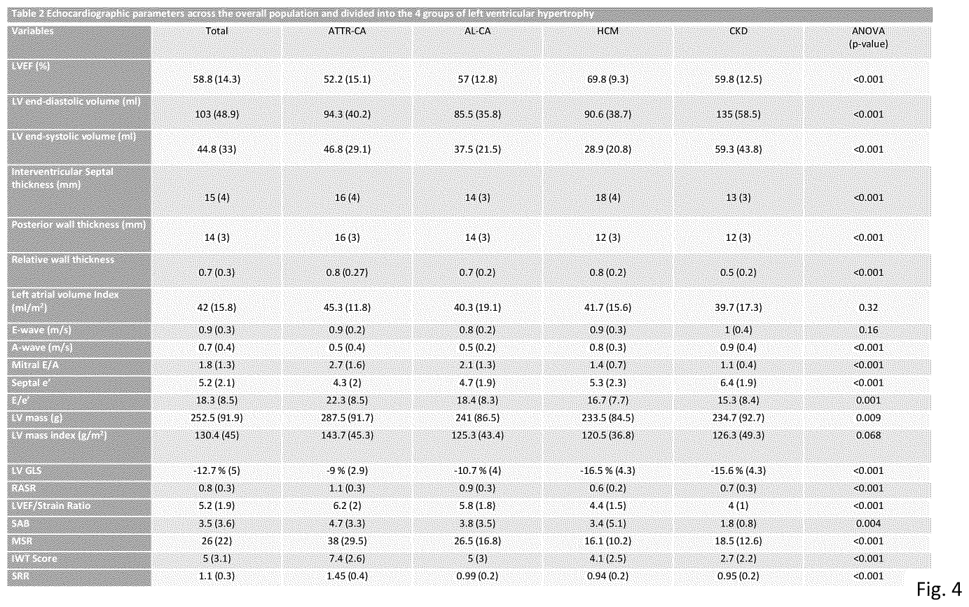

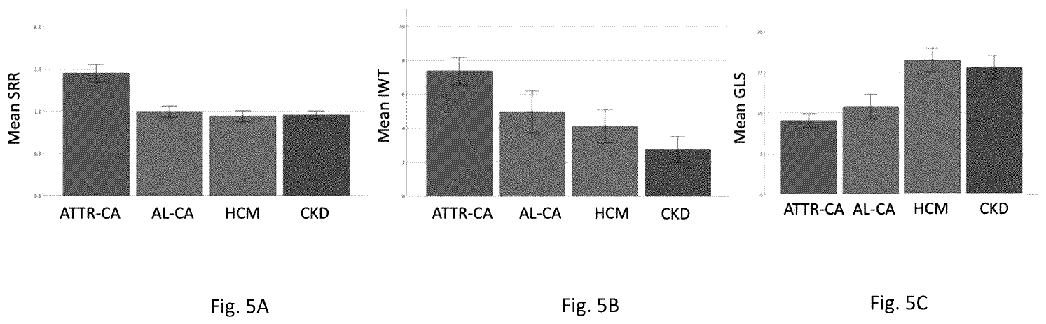

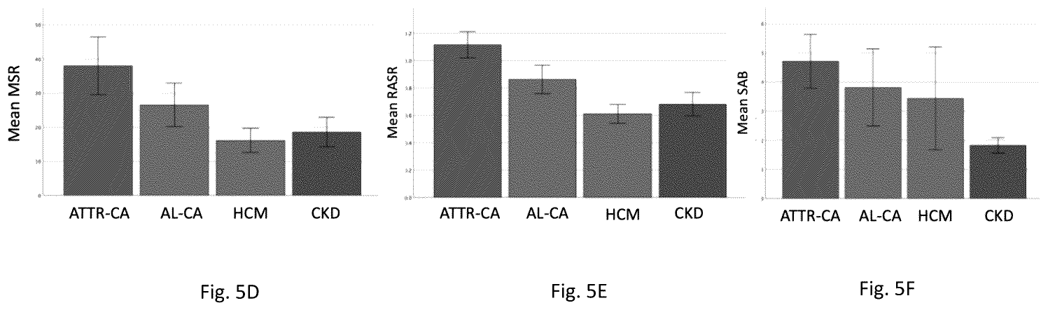

FIG. 4 is a table that depicts relevant echocardiography parameter findings across the 4 cohorts in accordance with an illustrative embodiment. FIG. 5A is a chart that depicts the mean septal reflectivity ratio for the 4 cohorts in the study in accordance with an illustrative embodiment. FIG. 5B is a chart that depicts the mean increased wall thickness (IWT) for the 4 cohorts in the study in accordance with an illustrative embodiment. FIG. 5C is a chart that depicts the mean global longitudinal strain (GLS) for the 4 cohorts in the study in accordance with an illustrative embodiment. FIG. 5D is a chart that depicts the mean mass to strain ratio (MSR) for the 4 cohorts in the study in accordance with an illustrative embodiment. FIG. 5E is a chart that depicts the mean relative apical sparing ratio (RASR) for the 4 cohorts in the study in accordance with an illustrative embodiment. FIG. 5F is a chart that depicts the mean septal apical to base ratio (SAB) for the 4 cohorts in the study in accordance with an illustrative embodiment.

As shown in FIGS. 4-5, the ATTR-CA cohort had significantly lower LVEF of 52.2% compared to the other cohorts, with the HCM patients having the highest LVEF of 69.5% (p=<0.001). The advanced CKD cohort had on average the highest mean end-diastolic volume (EDV) (135+/−58.5 mL) compared to the remaining cohorts' mean EDVs (ranging from 85.5-94.3 mL (p=<0.001)). Inter-ventricular septal (IVS) width varied across cohorts with HCM patients having the largest mean IVS width of 18 mm and advanced CKD with the lowest of 13 mm (p=<0.001). Posterior wall thickness (PWT) ranged from 12 mm in the HCM and advanced CKD cohorts to 16 mm in the ATTR-CA cohort (p=<0.001). Overall left atrial volume index was not significantly different across the groups. E/A ratios were the highest in the ATTR-CA cohort and lowest in the advanced CKD cohort. The mean LV mass index was highest in the ATTR-CA cohort at 143.7 g/m² (p=<0.001). In terms of speckle-tracking parameters and indices, the mean LV GLS was significantly lower in the ATTR-CA cohort at −9% and was highest in the HCM cohort at −16.5% (p=<0.001). The relative longitudinal apical sparing ratio, LVEF/strain ratio, mass to strain ratio, IWT score, and septal apical to base ratio were all highest in the ATTR-CA cohort compared to the other groups (p=<0.001). The mean septal reflectivity ratio (SRR) was significantly higher in the ATTR-CA cohort compared to the other cohorts (p=<0.001).

The data presented in FIGS. 4 and 5 illustrate the diagnostic value of the SRR against other echocardiographic indices for determining the presence of ATTR-CA amongst all groups. The receiver operator characteristic (ROC) curve analysis was utilized to determine the diagnostic value of traditional echocardiographic parameters, speckle-tracking derived indices, and the SRR in detecting the presence of ATTR-CA when compared against all groups. The SRR retained the largest AUC of 0.91 (p<0.001) with the relative apical sparing ratio (AUC=0.85; p<0.001) and LV GLS (AUC=0.84; p<0.001) having the next largest AUCs. Delong's test was not significant for the AUC difference between SRR, RASR and LV GLS. However, these 3 parameters were significantly different and discriminated better than the remaining chosen echocardiographic parameters for ATTR-CA compared to all other phenocopy cohorts.

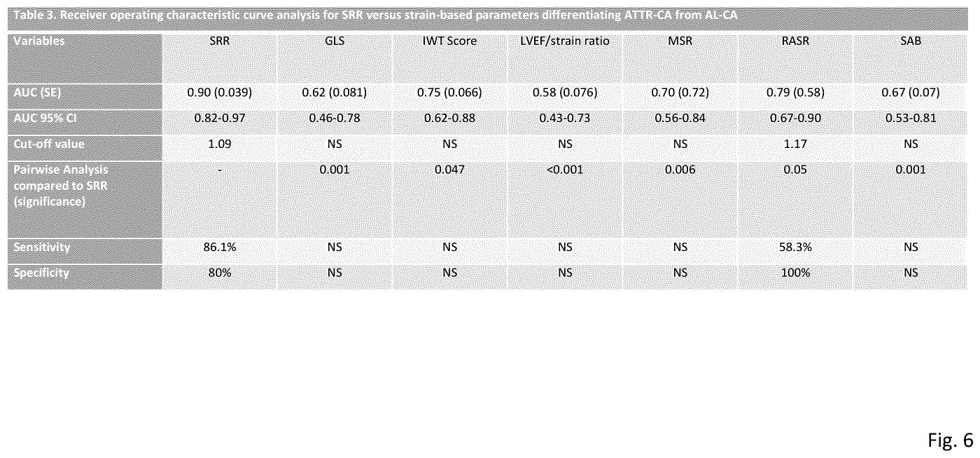

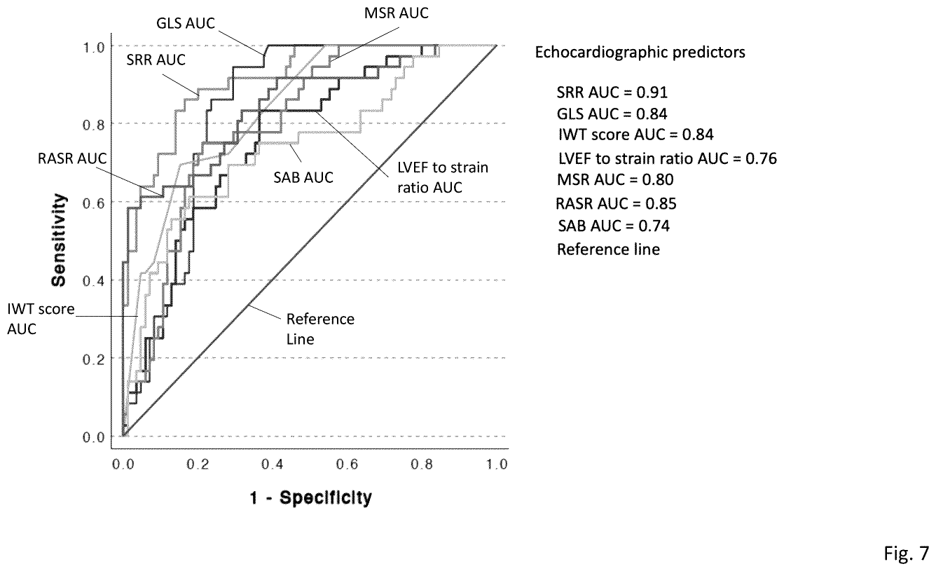

FIG. 6 is a table that depicts receiver operating characteristic curve analysis for SRR versus strain-based parameters differentiating ATTR-CA from AL-CA in accordance with an illustrative embodiment. FIG. 7 is a graph that depicts ROC curves for differentiating ATTR-CA from all other cohorts and that compares SRR against other important strain-based parameters in accordance with an illustrative embodiment. As discussed, this analysis shows that the SRR retains the largest AUC compared to other parameters but was not significantly higher than RASR or left ventricular GLS.

However, when the analysis was confined to utilizing the SRR compared to other echocardiographic parameters to discriminate between only the ATTR-CA and AL-CA cohorts, the SRR retained the largest AUC of 0.90 (p<0.0001) and Delong's test demonstrated a significant difference between this AUC and all other AUCs derived from other echocardiographic parameters (p<0.02). The optimal SRR based on Youden's index was 1.09, resulting in a sensitivity of 86.1% and a specificity of 80%. However, an SRR of 1.23 was found to be the optimal specific value for discriminating between ATTR-CA and AL-CA with a specificity of 96.4% and sensitivity of 63.2%, and a satisfactory Youden's index of 0.6. The LV mass to strain ratio (MSR) was inferior to the SRR in discriminating ATTR-CA from AL-CA (AUC 0.67 vs 0.88; p=0.003). After considering only patients that had complete data available for the IWT score (42 ATTR-CA, 25 AL-CA), the SRR still maintained superiority in discriminating between ATTR-CA from AL-CA (IWT AUC 0.73 vs SRR AUC 0.91; p=0.013).

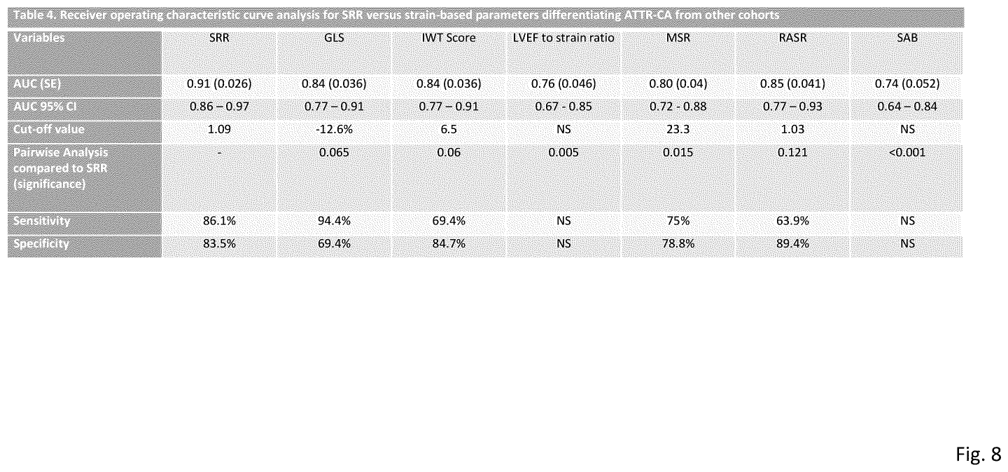

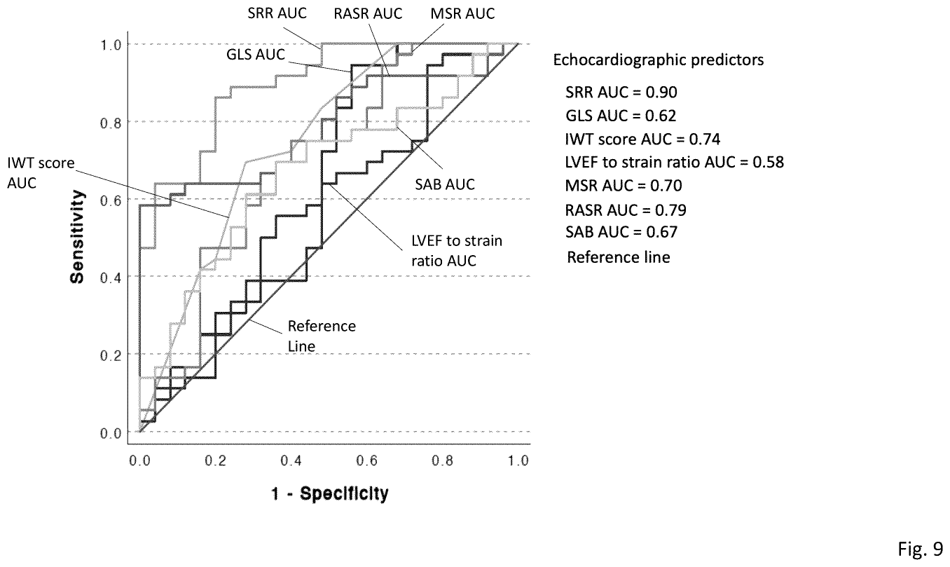

FIG. 8 is a table that depicts receiver operating characteristic curve analysis for SRR versus strain-based parameters differentiating ATTR-CA from other cohorts in accordance with an illustrative embodiment. FIG. 9 is a graph that depicts ROC curves for differentiating ATTR-CA from AL-CA by comparing SRR against other important strain-based parameters in accordance with an illustrative embodiment. As discussed, this analysis shows that SRR is superior to other strain-based parameters for differentiating ATTR-CA from AL-CA.

The reproducibility of the above-discussed results was also confirmed. Specifically, analysis of the SRR was performed on the same still frames as the original measurements by two blinded operators on 30 subjects randomly selected from across the 4 groups. Intraclass correlation coefficients were used to quantify interobserver reproducibility. There was excellent inter- and intra-operator reproducibility with an ICC of 0.91 (p<0.001) and 0.89 (p<0.001), respectively.

This is the first echocardiographic analysis utilizing novel pixel intensity quantification software to demonstrate a technique that can differentiate ATTR-CA from AL-CA. There are several important findings that resulted from the above-discussed study. One finding is that a SRR between the anterior septal and posterior lateral left ventricular walls, in the parasternal long axis view, of >1.23 is suggestive of a diagnosis of ATTR-CA over AL-CA when there is a suspicion of CA. Another finding is that the SRR is superior to other strain-based measures in differentiating ATTR-CA from AL-CA. It was also found that the SRR is equivalent to the relative apical sparing ratio in differentiating ATTR-CA from other common phenocopies of CA. Additionally, the SRR is reproducible and easily implemented for retrospective analysis of echocardiograms with the suspicion of CA.

As discussed above, CA is an infiltrative cardiomyopathy that is associated with significant morbidity and mortality. Determining the etiology of CA is intimately linked with the trajectory of patient outcomes. The most pertinent delineation in subtypes of CA is between ATTR-CA and AL-CA due to the effective but mechanistically divergent therapies available. Previous studies have sought to identify a non-invasive diagnostic test with the ability to accurately discriminate ATTR-CA from AL-CA. Using traditional techniques, the most reliable non-invasive diagnostic test to differentiate these two CA subtypes is a nuclear cardiology scan incorporating calcium-binding tracers such as the technetium-PYP scan.

However, the mechanisms underlying the affinity of ATTR-CA for calcium-binding bone markers are not fully understood. The presence of microcalcifications has been suggested as a possible mechanism. In a previous study, pathologic examination of endomyocardial biopsies from both ATTR-CA and AL-CA demonstrated significantly more calcium particles in ATTR-CA patients. Additionally, whole heart examination of patients with both ATTR-CA and AL-CA have established the presence of clusters of microcalcification more frequently in ATTR-CA. Furthermore, these calcium clusters tend to aggregate in the left ventricle, and particularly in the interventricular septum as a gradient from base to apex. This base to apical gradient distribution (i.e. relative apical sparing) in CA, and in particular ATTR-CA, has been demonstrated numerous times with multimodal imaging.

Although nuclear cardiology calcium-binding bone markers have been thoroughly studied for diagnosing ATTR-CA and differentiating it from AL-CA, there still lies an unmet need for a non-invasive (echocardiographic) differentiation of underlying CA subtypes. Successful echocardiographic differentiation could offer the potential promise of bypassing additional testing, avoiding radiation, and being a more widely available test compared to amyloid-specific nuclear imaging.

Given the propensity of microcalcifications to the LV and especially the interventricular septum in ATTR-CA, the inventors developed an echocardiographic method to exploit this calcification pattern and use this to distinguish ATTR-CA from AL-CA and other causes of left ventricular hypertrophy. It has been shown that pixel intensity (a measure of image brightness) from ultrasound images of plated calcium correlates strongly to both calcium weight and CT calcium score. Additionally, recent studies have demonstrated that pixel intensity quantification of the aortic valve from echocardiographic images (using ImageJ software) correlates strongly with aortic valve calcification (AVC) determined by multidetector computed tomography (MDCT) and echocardiographic hemodynamic quantification of AS. Furthermore, pixel-intensity AVC quantification can incrementally differentiate between all severities of AS.

Considering the established evidence for detecting calcium echocardiographically with manual annotation, the inventors applied a similar method in the CA cohort, as discussed above with reference to FIG. 1. Accounting for previous pathologic studies demonstrating an inclination for microcalcifications in the septum in ATTR-CA patients, the pixel intensity of the left ventricular anterior septal wall was measured relative to the contralateral posterior lateral wall to differentiate ATTR-CA from AL-CA and other causes of left ventricular hypertrophy. It was hypothesized that this view would accentuate any differences between the two forms of CA resulting in lower pixel intensity quantification of the posterior lateral wall (and thus higher SRR) in ATTR-CA patients due to increased far field shadowing from microcalcifications in the anterior septum (see FIG. 1).

Indeed, the above-discussed study did show a markedly higher SRR in patients with ATTR-CA compared to AL-CA (1.45 vs 0.99, p=<0.001), hypertrophic cardiomyopathy (1.45 vs 0.94, p=<0.001), and advanced CKD (1.45 vs 0.95, p=<0.001). Additionally, SRR retained the largest AUC for differentiating ATTR-CA from all other causes of left ventricular hypertrophy (AUC=0.91; p=<0.001) and specifically for differentiating ATTR-CA from AL-CA (AUC=0.89; p=<0.001). It is noted, however, that ATTR-CA is not unique in its propensity for myocardial calcium deposition. Various causes of myocardial calcification have been reported, ranging from traumatic, infarction, infectious, inflammatory, and neoplastic. Even AL-CA has been reported to have a variable degree of myocardial microcalcification deposition. However, AL-CA has not been reported to have a relative predilection for septal myocardial microcalcifications which likely explains why the SRR was able to exploit relatively elevated hyperechoic anterior septal wall reflectivity and accurately distinguish ATTR-CA from AL-CA in the study. Additionally, the most common cause of myocardial calcification is myocardial infarction. Although this may confound the SRR measurement, it is noted that none of the patients in the study had evidence of prior myocardial infarction within the septal distribution.

Traditional and contemporary echocardiographic parameters have to date had mixed success in being able to differentiate ATTR-CA from AL-CA. Techniques such as speckle-tracking parameters have found success in differentiating CA from other forms of left ventricular hypertrophy. However, in classifying between CA subtypes, individual measures have either performed poorly or failed altogether. The relative apical sparing ratio (RASR) was originally discovered to differentiate CA from other LVH phenocopies. The SRR had a larger AUC compared to this index when differentiating ATTR-CA from other phenocopies, but when classifying ATTR-CA from AL-CA, the SRR performed significantly better than the RASR. Alternatively, others have explored the performance of multi-parametric echocardiographic scores whereby traditional measures of LV mass, diastolic function, and speckle tracking echocardiography were incorporated to generate the IWT score. Although the IWT was able to diagnose CA within the excellent range in a cohort of patients with suspected CA, it was not superior to the singular LV mass to strain ratio (MSR). In the present study the MSR was only able to modestly differentiate ATTR-CA from AL-CA (AUC 0.74). Additionally, the IWT score utilizes the combined weight of multiple parameters, all of which if measured incorrectly could significantly affect the diagnostic accuracy of the score. More importantly, the SRR performed significantly better than the LV MSR (AUC 0.90 vs 0.70; p<0.001) and IWT (AUC 0.75, p=0.047) in discriminating between ATTR-CA and AL-CA in the present cohort.

Cardiac MRI has provided an alternative modality for further delineating ATTR-CA from AL-CA. The ability to assess extra-cellular volume and late gadolinium enhancement (LGE) patterns specific to ATTR-CA provides a degree of anatomic tissue characterization unavailable to existing echocardiographic parameters. A comprehensive meta-analysis found excellent specificity and good sensitivities for subendocardial LGE patterns in the left ventricle and left atria as markers of cardiac amyloidosis compared to phenocopies. However, like nuclear studies, cardiac MRI requires warrants additional testing that suffers from fiscal and availability issues, requires administration of gadolinium, and specialized readers compared to echocardiography which is more ubiquitous and cheaper.

In practice, to detect the probability of ATTR amyloid, a series of analyses are conducted on echocardiographic data, which can exist in various image or video format(s). In an illustrative embodiment, an optimal view is determined manually or automatically and is the view that best showcases a majority of the septum, ventricular wall, and ventricular chamber and is a critical step in preprocessing. This image/video is eventually passed to a fully trained machine learning (ML) or artificial intelligence (AI) algorithm that is capable of discerning different facets of the image/video automatically.

Using ML/AI, the proposed algorithm is designed to identify several different entities in the image/video to ultimately enable automatic detection of the regions of interest (ROI) needed to calculate the SRR. Training this algorithm effectively, hinges on human input. This can come in the form of prompts, manual annotations, or a combination of the two. The algorithm can be trained in a completely supervised fashion or using self-supervised learning (SSL) employing few-shot or zero-shot learning on the specific task. Semantic segmentation using the algorithm will identify regions in the image from multiple classes of semantically interpretable structures. Following training, future segmentation of ROIs occurs in an automatic fashion, attempting to reproduce what manual annotation achieved during development/validation outlined earlier. Using the algorithm, data input is encoded to arrive at a latent space representation.

In an illustrative embodiment, a decoder can be used to convert the latent space into semantic segmentation. This will employ the use of a mask head leveraging convolutional layers to up-sample and generate a binary mask of the various ROIs. The semantically interpretable structures can include but are not limited to the septum, ventricular wall, and ventricle space from an echocardiogram. These identified regions of interest (ROIs) will serve as the backbone of the computation utilized to perform the ratio of intensities seen between the septum and the ventricular wall. The intensities present in these two ROIs can both be standardized to the overall gain present in the image by employing a 3rd ROI (e.g., the ventricular chamber) and other facets of the image/video (i.e. the histogram of intensities) present. The SRR measure may also ultimately be combined into additional or downstream architectures and data types/modalities for multimodal learning in ascertaining a disease diagnosis pertaining to ATTR-CA.

The fully trained ML model can take less than 5 megabytes (Mb) of storage space. However, it is the subsequent computations on a given image that can potentially make the ML algorithm computationally expensive. For that reason, a system with >16 gigabytes (Gb) of random-access memory (RAM) should be used. The CUDA programming language can be used to parallelize operations if a NVIDIA graphical processing unit (GPU) is available on the deployed machine/cloud. Otherwise, operations will be performed using a central processing unit (CPU). This means that if a GPU is available, computations can occur in parallel with respect to the GPU and asynchronously with respect to the CPU.

The software can be operating system agnostic, and Python can be used to create the algorithms used to perform the analysis and calculations. However, this may be adapted to use a compiled language (e.g. C++, Java, etc.) to integrate and operate within existing frameworks by companies for whom licenses may be granted. It is expected that a compiled or just in time (jit) compilation version of the software that has a graphical user interface (GUI) will be how end users will interact with the proposed system. It is envisioned that this software will be an integrated part of other GUI based software used in the manual assessment of echocardiograms, performed by cardiologists. Interaction for running the model can constitute a drop-down menu or additional button in the GUI a given clinician could click to run the analysis. Computations may proceed locally or in cloud-based environment.

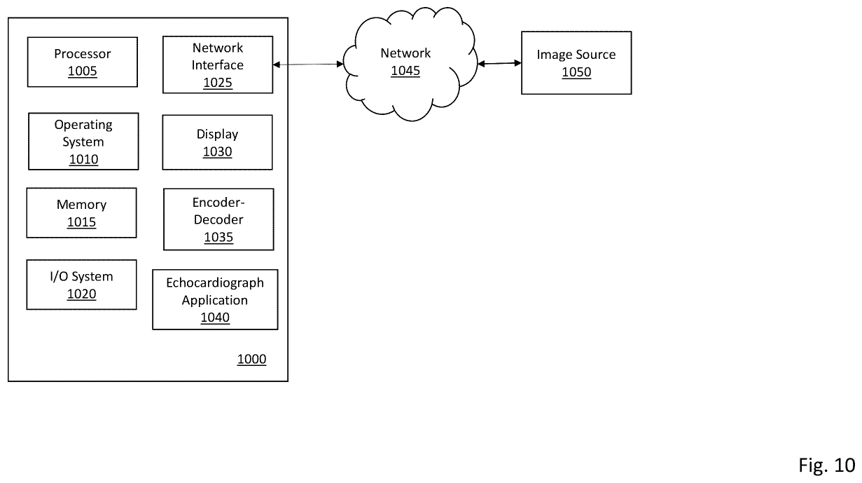

FIG. 10 is a block diagram of components included in an echocardiographic detection system for distinguishing subtypes of cardiac amyloidosis in accordance with an illustrative embodiment. The detection system includes a computing device 1000 which includes a processor 1005, an operating system 1010, a memory 1015, an input/output (I/O) system 1020, a network interface 1025, a display 1030, an encoder/decoder 1035, and an echocardiograph application 1040. In alternative embodiments, the computing device 1000 may include fewer, additional, and/or different components. The components of the computing device 1000 communicate with one another via one or more buses or any other interconnect system. In an illustrative embodiment, the computing device 1000 can be a desktop computer, a laptop computer, a dedicated computing system, a tablet, a smart phone, etc.

The processor 1005 of the computing device 1000 can be used alone or in conjunction with the echocardiograph application 1040 to perform any of the operations described herein, such as obtaining image data, processing the image data, making diagnostic determinations regarding the processed data, sending data and/or results to external systems, etc. The processor 1005 can be any type of computer processor known in the art and can include a plurality of processors and/or a plurality of processing cores. The processor 1005 can include a controller, a microcontroller, an audio processor, a graphics processing unit, a hardware accelerator, a digital signal processor, etc. Additionally, the processor 1005 may be implemented as a complex instruction set computer processor, a reduced instruction set computer processor, an x86 instruction set computer processor, etc. The processor 1005 is used to run the operating system 1010, which can be any type of operating system.

The operating system 1010 is stored in the memory 1015, which is also used to store programs, algorithms, network and communications data, peripheral component data, the echocardiograph application 1040, and other operating instructions. The memory 1015 can be one or more memory systems that include various types of computer memory such as flash memory, random access memory (RAM), dynamic (RAM), static (RAM), a universal serial bus (USB) drive, an optical disk drive, a tape drive, an internal storage device, a non-volatile storage device, a hard disk drive (HDD), solid state drive (SDD), a volatile storage device, etc.

The I/O system 1020, or user interface, is the framework which enables users (and peripheral devices) to interact with the computing device 1000. The I/O system 1020 can include one or more keys or a keyboard, one or more buttons, a speaker, a microphone, etc. The I/O system 1020 allows the user to interact with and control the computing device 1000. The I/O system 1020 can also include circuitry and a bus structure to interface with and control peripheral computing components such as one or more power sources, etc. The computing device 1000 also includes a display 1030, which can be part of the I/O system 1020. The display 1030 can be used to output and display images for user assessment, calculated data, determined diagnoses, operating instructions, operating controls, other GUIs, etc. Any type of display can be used, including a liquid crystal display (LCD), a light-emitting diode (LED) display, a touchscreen display, etc. In one embodiment, the I/O system 1020 can also include a printer that is used to print any of the images and/or results described herein.

The network interface 1025 includes transceiver circuitry (e.g., a receiver and/or a transmitter) that allows the computing device 1000 to transmit and receive data to/from other devices such as an image source 1050. The network interface 1025 enables communication through a network 1045, which can be one or more communication networks. The network 1045 can include a cable network, a fiber network, a cellular network, a wi-fi network, a landline telephone network, a microwave network, a satellite network, etc. The network interface 1025 also includes circuitry to allow device-to-device communication such as near field communication (NFC), Bluetooth® communication, etc. In alternative embodiments, the computing device 1000 may be a standalone system that does not connect to the network 1045.

In an illustrative embodiment, the image source 1050 can be a database or other storage/memory repository in which echocardiographic images are stored and accessed by the computing system 1000. As discussed, the images can be accessed through the network 1045. Alternatively, the images can be directly accessed by the computing system 1000 without the use of a network. In another alternative embodiment, the image source 1050 can be an imaging system that generates the echocardiographic images, such as an ultrasound imaging system, a magnetic resonance imaging (MRI) system, etc.

The encoder/decoder 1035 is used to facilitate semantic segmentation. The semantic segmentation is used to identify regions in a received image from one or more classes of semantically interpretable structures. As discussed above, these layers include several convolutional, pooling, and batch normalizations in the encoder to arrive at a latent space representation. The decoder includes a series of pooling layers, up-sampling layers, and a full connected layer at the terminal point to provide a semantic segmentation from the latent space representation of the echocardiogram image. The semantically interpretable structures include the septum, ventricular wall, and ventricle space from the echocardiogram, which are used as described herein to determine the septal reflectivity ratio.

The echocardiograph application 1040 can include software and algorithms (e.g., in the form of computer-readable instructions) which, upon activation or execution by the processor 1005, performs any of the various operations described herein such as receiving image data, extracting relevant data from the received image data, analyzing the extracted data to determine the septal reflectivity ratio, making a diagnosis based on the analysis, communicating with remote computing devices, etc.

More specifically, the echocardiograph application 1040 automatically analyzes echocardiographic still frames of the left ventricle in the parasternal long axis at end diastole. The anterior septal and posterior lateral myocardial walls are automatically detected in still frame echocardiographic images. The pixel intensity is measured as the brightness (or luminance) of the light emitted by a digital display The pixel intensity is measured within the automatically detected region(s) of interest corresponding to the anterior septal and posterior lateral myocardial walls at end diastole in the parasternal long-axis view. Pixel densities of the left ventricular blood pool immediately adjacent to both walls and in the RV blood pool are also automatically measured to ensure no significant differences in gain within each image at the depth of the anterior septal and posterior lateral walls. The anterior septal wall pixel intensity is divided by the posterior lateral wall pixel intensity to generate the septal reflectivity ratio (SRR). The echocardiograph application 1040 can also compare the SRR to a threshold value to determine the type of cardiac amyloidosis. In an illustrative embodiment, an SRR greater than 1.23 (i.e., the threshold value) is an accurate discriminator of ATTR-CA from AL-CA and other mimickers of ATTR-CA (e.g., hypertrophic cardiomyopathy and end stage renal disease). In alternative embodiments, a different SRR threshold may be used. The echocardiograph application 1040 can utilize the processor 1005 and/or the memory 1015 as discussed above.

In summary, it is known that subtypes of cardiac amyloidosis have widely divergent treatments and require early diagnostic differentiation. The inventors have found that ATTR-CA fibrils contain microcalcification clusters preferentially deposited in the ventricular septum. It was shown that acoustic shadowing from septal microcalcifications results in a higher septal reflectivity ratio in ATTR-CA than AL-CA and other cardiac amyloidosis phenocopies. The septal reflectivity ratio robustly differentiates ATTR-CA from other phenocopies of cardiac amyloidosis and specifically from AL-CA which can expedite management decisions in cardiac amyloidosis. Thus, SRR is a robust method for differentiating ATTR-CA from other phenocopies of CA and specifically ATTR-CA from AL-CA. This method may reduce the need for additional testing and radiation exposure, and is more widely available compared to amyloid-specific nuclear imaging.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A system to distinguish subtypes of cardiac amyloidosis, the system comprising:
a memory configured to store an echocardiographic image of at least a portion of a heart; and
a processor operatively coupled to the memory, wherein the processor is configured to:
analyze the echocardiographic image to identify a region of interest of the heart;
determine a septal reflectivity ratio of the region of interest; and
determine, based at least in part on the septal reflectivity ratio, a subtype of cardiac amyloidosis present in the region of interest.

2. The system of claim 1, wherein the echocardiographic image comprises a view of a left ventricle in the parasternal long axis.

3. The system of claim 2, wherein the view of the echocardiographic image is captured at end diastole.

4. The system of claim 1, wherein the region of interest is bounded by an anterior septal myocardial wall of the heart and a posterior lateral myocardial wall of the heart.

5. The system of claim 4, wherein the region of interest includes fibrils containing microcalcification clusters.

6. The system of claim 1, wherein the processor is further configured to determine a luminance of the region of interest.

7. The system of claim 6, wherein the processor is configured to determine a first pixel intensity of an anterior septal myocardial wall of the heart and a second pixel intensity of a posterior lateral myocardial wall of the heart based at least in part on the luminance.

8. The system of claim 7, wherein the processor divides the first pixel intensity by the second pixel intensity to determine the septal reflectivity ratio.

9. The system of claim 1, wherein the processor compares the septal reflectivity ratio to a threshold value to determine the subtype of cardiac amyloidosis.

10. The system of claim 9, wherein the threshold value is 1.23.

11. The system of claim 9, wherein the processor distinguishes between transthyretin cardiac amyloidosis and light chain cardiac amyloidosis based on the comparison to the threshold value.

12. A method to distinguish subtypes of cardiac amyloidosis, the method comprising:
receiving, at a memory of a computing device, an echocardiographic image of at least a portion of a heart;
analyzing, by a processor of the computing device, the echocardiographic image to identify a region of interest of the heart;
determining, by the processor, a septal reflectivity ratio of the region of interest; and
determining, by the processor and based at least in part on the septal reflectivity ratio, a subtype of cardiac amyloidosis present in the region of interest.

13. The method of claim 12, further comprising capturing the echocardiographic image such that the echocardiographic image includes a view of a left ventricle in the parasternal long axis.

14. The method of claim 13, wherein the echocardiographic image is captured at end diastole.

15. The method of claim 12, wherein determining the region of interest includes identifying a region of the heart that is bounded by an anterior septal myocardial wall of the heart and a posterior lateral myocardial wall of the heart.

16. The method of claim 12, wherein the region of interest includes fibrils containing microcalcification clusters.

17. The method of claim 12, further comprising determining, by the processor, a first pixel intensity of pixels corresponding to an anterior septal myocardial wall of the heart and a second pixel intensity of pixels corresponding to a posterior lateral myocardial wall of the heart.

18. The method of claim 17, further comprising dividing, by the processor, the first pixel intensity by the second pixel intensity to determine the septal reflectivity ratio.

19. The method of claim 12, further comprising comparing, by the processor, the septal reflectivity ratio to a threshold value to determine the subtype of cardiac amyloidosis.

20. The method of claim 19, further comprising distinguishing, by the processor, between transthyretin cardiac amyloidosis and light chain cardiac amyloidosis based on the comparison to the threshold value.
